(19) United States
(12) Patent Application Publication
(10) Pub. No.: US 2019/0114464 A1
(43) Pub. Date: Apr. 18, 2019

(54) METHOD OF CURVILINEAR SIGNAL DETECTION AND ANALYSIS AND ASSOCIATED PLATFORM

(71) Applicant: GENOMIC VISION, Bagneux (FR)

(72) Inventors: Jeremie NICOLLE, Paris (FR); Laurent TOURTE, Paris (FR); Ivan KYRGYZOV, L'Hay les Roses (FR); Aymen SHABOU, Le Plessis Rpbinson (FR); Olivier LEVREY, Brou sur Chantereine (FR); Vincent GLAUDIN, Sceaux (FR)

(73) Assignee: GENOMIC VISION, Bagneux (FR)

(21) Appl. No.: 16/083,311

(22) PCT Filed: Mar. 10, 2017
(86) PCT No.: PCT/IB2017/000356

§ 371 Date: Sep. 7, 2018

Related U.S. Application Data
(60) Provisional application No. 62/306,296, filed on Mar. 10, 2016.

(51) Int. Cl. G06K 9/00; G06K 9/32; G06T 7/00; G16B 30/10

(52) U.S. Cl. CPC G06K 9/00127; G06K 9/3233; G06T 7/0012; G06T 2207/30072; G06K 2209/05; G06K 2209/21; G16B 30/10

(57) ABSTRACT

The present invention is related to a method of identifying at least one sequence of target regions on a plurality of macromolecules to test, each target region being associated with a tag and said macromolecules having underwent linearization according to a predetermined direction, wherein said method comprises performing by a processor (11) of equipment (10) the following steps: (a) receiving from a scanner (2) being sensitive to said tags, at least one sample image depicting said macromolecules as curvilinear objects sensibly extending according to said predetermined direction; (b) Generating a binary image from the sample age; (c) For at least one template image, and for each sub-area of the binary image having the same size as the template image, calculating a correlation score between the sub-area and the template image; (d) For each sub-area of the binary image for which the correlation score with a template image is above a first given threshold, selecting the corresponding sub-area of the sample image; (e) For at least one reference code pattern, and for each selected sub-area of the sample image, calculating an alignment score between the sub-area and the reference code pattern, said reference code pattern being defined by a given sequence of tags; (f) For each selected sub-area of the sample image for which the alignment score with a reference code pattern is above a second given threshold, identifying each target region depicted in said selected sub-area among the target regions associated with the tags defining said reference code pattern; (g) Outputting the different sequence(s) of identified target regions.

### FIELD OF THE INVENTION

The present invention concerns the field of macromolecule analysis, in particular nucleic acids.

More precisely, it relates to a method of identifying at least one sequence of target regions on a plurality of macromolecules to test, a method of machine learning for filtering pertinent objects (sequences) and a method for detecting anomalies within said macromolecules.

### BACKGROUND

The study of the macromolecules, in particular biological ones (more specifically DNA), often requires to mark up precisely some domains, either for “cartographic” purposes, i.e. to study the spatial organization of these domains, or for the purpose of locating the position, on the macromolecule, of a reaction or a set of chemical or biochemical reactions.

The observation of spatial organization of DNA implies that some regions are landmarked, i.e. marked in a way that allows identification of specific regions through some detection technique. This is the case for cartographic applications, where the main issue addressed is the relative position of several regions, as well as applications where a biological phenomenon is studied in one (several) specific locus (loci). Domains can then be identified by specific markers such as “probes”, i.e. sequences complementary to the regions of interest, coupled to “tags” which allow detection (fluorochromes of different colors, radioelements, etc.).

In particular, molecular combing is a technique used to produce an array of uniformly stretched DNA that is then highly suitable for nucleic acid hybridization studies such as fluorescent in situ hybridisation (FISH) which benefit from the uniformity of stretching, the easy access to the hybridisation target sequences, and the resolution offered by the large distance between two probes.

After molecular combing, are obtained large raw images in which DNA strands appear as numerous curvilinear objects extending sensibly according to a same direction (the combing direction), see the example of FIG. 1.

Image analysis allows detecting the DNA strands (as curvilinear objects) and distinguishing probes from noisy background (and identifying various kinds of probes).

Tags could be chosen so as to form a readable “code” defining a signature of the domains of interest, as proposed by the applicant in the international application WO 2008028931, which is here incorporated by reference.

Given an image of N×N pixels, the number of possible line segments defined is in O(N⁴). Direct evaluation of line integrals upon the whole set of segments is practically infeasible due to the computational burden. One of the methodologies proposed to address this problem is the Beamlet transform, as described in the international application WO 2008125663.

It defines a set of dyadically organized line segments occupying a range of dyadic locations and scales, and spanning a full range of orientations. This system of line segments, called beamlets, have both their end-points lying on dyadic squares that are obtained by recursive partitioning of the image domain. The collection of beamlets has a O(N² log(N)) cardinality. The underlying idea of the Beamlet transform is to compute line integrals only on this smaller set, which is an efficient substitute of the entire set of segments for it can approximate any segment by a finite chain of beamlets. Beamlet chaining technique also provides an easy way to approximate piecewise constant curves.

Formally, given a beamlet b=(x, y, l, θ) centered at position (x,y), with a length l and an orientation θ, the coefficient of b computed by the Beamlet transform is given by:

$$Φ ( f , b ) = {\int }_{- l / 2}^{l / 2} f ( x + γ cos ( θ ) , y + γ sin ( θ ) ) d γ .$$

Peaks in the parameter space reveals potential lines of interest. This is a very reliable method for detecting lines in noisy images, but still requires high performance computational equipment, as input raw images typically contain over one billion of pixels, for a size of several gigabytes.

It would be useful to provide a new method for detecting curvilinear objects of an image, which would be even more efficient and reliable, so as to allow rapid detection of macromolecules and identification of their spatial organization in very large raw images.

After detecting curvilinear objects it would be useful to provide a new method of machine learning in order to get the most pertinent candidates of objects that could be used farther for anomaly detection or any type of analysis. Such a method can also provide a ranking of objects relative to their informative pertinence.

Furthermore, it would be useful to provide a new method for analyzing such spatial organization of macromolecules so as to easily detect and identify any anomaly therein.

### SUMMARY OF THE INVENTION

The invention proposes according to a first aspect a method of identifying at least one sequence of target regions on a plurality of macromolecules to test, each target region being associated with a tag and said macromolecules having underwent linearization according to a predetermined direction, wherein said method comprises performing by a processor of equipment the following steps:

(a) receiving from a scanner being sensitive to said tags, at least one sample image depicting said macromolecules as curvilinear objects sensibly extending according to said predetermined direction;

(b) Generating a binary image from the sample image;

(c) For at least one template image, and for each sub-area of the binary image having the same size as the template image, calculating a correlation score between the sub-area and the template image;

(d) For each sub-area of the binary image for which the correlation score with a template image is above a first given threshold, selecting the corresponding sub-area of the sample image;

(e) For at least one reference code pattern, and for each selected sub-area of the sample image, calculating an alignment score between the sub-area and the reference code pattern, said reference code pattern being defined by a given sequence of tags;

(f) For each selected sub-area of the sample image for which the alignment score with a reference code pattern is above a second given threshold, identifying each target region depicted in said selected sub-area among the target regions associated with the tags defining said reference code pattern;

(g) Outputting the different sequence(s) of identified target regions.

In an embodiment, each target region is bound to a molecular marker, itself labelled with a tag.

In an embodiment, the macromolecule is nucleic acid, particularly DNA, more particularly double strand DNA.

In an embodiment, the molecular markers are oligonucleotides probes.

In an embodiment, linearization of the macromolecule is performed by molecular combing or Fiber Fish.

In an embodiment, said tags are fluorescent tags.

In an embodiment, the target regions are associated with at least two different tags.

In an embodiment, step (a) comprises, for a field of view of the scanner, receiving from the scanner a sample image of the field of view for each tag.

In an embodiment, step (b) comprises generating a binary image for each sample image, and merging the binary images from sample images of the same field of view.

In an embodiment, said alignment score is computed using correlation method.

In an embodiment, generating a binary image at step (b) comprises applying a local mean thresholding filter according to a direction which is orthogonal to said predetermined direction.

In an embodiment, the method further comprises a step (b′) of post-processing the generated binary image so as to remove unnecessary information.

In an embodiment, the templates images of step (c) represent the same object according to different orientations.

In an embodiment, said objet is a segment.

In an embodiment, said different orientations are around said predetermined orientation.

In an embodiment, step (d) comprises applying on the selected sub-areas a thresholding filter using machine learning algorithms.

According to a second aspect is proposed a method of identifying at least one sequence of target regions on a plurality of macromolecules to test, each target region being associated with a tag and said macromolecules having underwent linearization according to a predetermined direction, wherein said method comprises performing by a processor of equipment the following steps:

(α) receiving a plurality of candidate sub-areas of a sample image from a scanner being sensitive to said tags, each sub-area possibly depicting one of said macromolecules as a curvilinear objects sensibly extending according to a predetermined direction;

(β) applying on the candidate sub-areas a thresholding filter using machine learning algorithms so as to select relevant sub-areas;

(χ) For at least one reference code pattern, and for each selected sub-area, calculating an alignment score between the sub-area and the reference code pattern, said reference code pattern being defined by a given sequence of tags;

(δ) For each selected sub-area of the sample image for which the alignment score with a reference code pattern is above a second given threshold, identifying each target region depicted in said selected sub-area among the target regions associated with the tags defining said reference code pattern;

(ϵ) Outputting the different sequence(s) of identified target regions.

According to a third aspect is proposed an equipment comprising a processor implementing:

A module for receiving from a scanner connected to said equipment, at least one sample image depicting macromolecules to test as curvilinear objects sensibly extending according to a predetermined direction, said macromolecules presenting at least a sequence of target regions, each target region being associated with a tag and said macromolecules having underwent linearization according to said predetermined direction, wherein said method;
A module for generating a binary image from the sample image;
A module for calculating, for at least one template image, and for each sub-area of the binary image having the same size as the template image, a correlation score between the sub-area and the template image;
A module for selecting, for each sub-area of the binary image for which the correlation score with a template image is above a first given threshold, the corresponding sub-area of the sample image;
A module for calculating, for at least one reference code pattern, and for each selected sub-area of the sample image, an alignment score between the sub-area and the reference code pattern, said reference code pattern being defined by a given sequence of tags;
A module for identifying, for each selected sub-area of the sample image for which the alignment score with a reference code pattern is above a second given threshold, each target region depicted in said selected sub-area among the target regions associated with the tags defining said reference code pattern;
A module for outputting the different sequence(s) of identified target regions.

It is to be understood that both the foregoing general description of the invention and the following detailed description are exemplary, but are not restrictive, of the invention.

### BRIEF DESCRIPTION OF THE DRAWINGS

The above and other objects, features and advantages of this invention will be apparent in the following detailed description of an illustrative embodiment thereof, with is to be read in connection with the accompanying drawings wherein:

FIG. 1 represents an example of a sample image depicting macromolecules to test;

FIG. 2 represents an architecture of system for performing the methods according to the invention;

FIG. 3 illustrates an example of division a large image into tiles;

FIG. 4 represents an example of filter for generating a binary image;

FIG. 5 represents different binary channels generated and combined for the example of sample image of FIG. 1;

FIG. 6a represents examples of template images;

FIG. 6b illustrates a possible path of a template image within the sample image;

FIG. 6c illustrate a possible machine learning framework for validation;

FIG. 7a represents an example of reference code pattern;

FIG. 7b illustrates a possible path of a reference code pattern within the sample image;

FIG. 7c represents an example of selected sub-area of the sample image along with corresponding code pattern;

FIG. 7d represents an example of sequence detection without machine learning approach (o—true positive signals, x—false positive signals):

FIG. 7e represents an example of significantly improved signal detection after applying the machine learning method (o—true positive signals, x—false positive signals);

FIG. 8 represents example of anomalies to be detected;

FIG. 9a represents the example of reference code pattern of FIG. 7a with labelled gaps;

FIG. 9b illustrates with examples the different cases of gap labelling rules;

FIG. 10 represents a preferred embodiment of a step of determining if a target region presents a bimodal distribution of length;

FIG. 11 represents an example of an output report.

### DETAILED DESCRIPTION

### First and Second Mechanisms

The present invention concerns two complementary and independent mechanisms that will be successively described.

The first mechanism is related to two methods of identifying at least one sequence of target regions on a plurality of macromolecules to test.

The second mechanism is related to a method of analyzing a sequence of target regions on a plurality of macromolecules to test (in particular identified according to the first mechanism) so as to detect anomalies therein.

### Preparation of Macromolecules

Said macromolecules to test, which are preferably nucleic acid, particularly DNA, more particularly double strand DNA (in the case of molecular combing is used for linearization of the DNA), but which can also be proteins, polymers, carbohydrates or other types of molecules consisting of one or more long chains of basic elements, present domains of interest, which are defined as a sequence of target regions, said target regions being previously bound with specific complementary molecular marker (such as hybridization probes for nuclear acid) so as to “prepare” the macromolecules for testing.

As already explained, a probe is typically a fragment of DNA or RNA of variable length.

In an embodiment, the probes are oligonucleotides of at least 15 nucleotides, preferably at least 1 Kb more preferably between 1 to 10 kb, even more preferably between 4 to 10 kb.

Each probe thereby hybridizes to single-strand nucleic acid (DNA or RNA) whose base sequence allows base pairing between the target region and the probe due to complementarity. The probe is first denatured (by heating or under alkaline conditions such as exposure to sodium hydroxide) into single strand DNA (ssDNA) and then hybridized to the target region.

A specific molecular marker (such as a probe) is itself labelled with a “tag” or “label”, i.e. a molecule or an atom able to be detected by suitable optical sensors, such as a fluorescent molecule.

The sequence of molecular makers (nature and the position of markers) as identified thanks to their tags defines a “signature” of the macromolecule to test.

In the present description, the preferred example of nucleic acid strands hybridized with fluorescent probes will be detailed, but it has to be understood that any kind of molecular marker able to bind to the macromolecule to test (for example, antibodies if the macromolecule is a protein), labelled with any tag. The skilled person will know how to adapt the invention.

Detectable tags suitable for use in the present invention include any composition detectable by spectroscopic, photochemical, electrical or optical means. Useful tags in the present invention include biotin for staining with labelled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radioisotopes (e.g., .³H, ¹²⁵I, ³⁵S, ¹⁴C, or .³²P), enzymes (e.g., horse radish peroxidase, alkaline phosphatase and others commonly used in an ELISA), and colorimetric tags such as colloidal gold (e.g., gold particles in the 40-80 nm diameter size range scatter green light with high efficiency) or colored glass or plastic (e.g., polystyrene, polypropylene, latex, etc.) beads.

A fluorescent tags is preferred because it provides a very strong signal with low background. It is also optically detectable at high resolution and sensitivity through a quick scanning procedure.

The tags may be incorporated by any of a number of means well known to those of skill in the art. However, in a preferred embodiment, the tags are simultaneously incorporated during the amplification step in the preparation of the molecular markers. For example, polymerase chain reaction (PCR) with labelled primers or labelled nucleotides will provide a labelled amplification product. The probe (e.g., DNA) is amplified in the presence of labelled deoxynucleotide triphosphates (dNTPs).

In a preferred embodiment, transcription amplification, as described above, using a labelled nucleotide (e.g. fluorescein-labelled UTP and/or CTP) incorporates a tag into the transcribed nucleic acids.

Alternatively, a tag may be added directly to the original probe (e.g., mRNA, polyA mRNA, cDNA, etc.) or to the amplification product after the amplification is completed. Such labelling can result in the increased yield of amplification products and reduce the time required for the amplification reaction. Means of attaching tags to probes include, for example nick translation or end-labelling (e.g. with a labelled RNA) by kinasing of the nucleic acid and subsequent attachment (ligation) of a nucleic acid linker joining the probe to a tag (e.g., a fluorophore).

Preferably, labelled nucleotides according to the present invention are Chlorodeoxyuridine (CIdU), Bromoeoxyuridine (BrdU) and or lododeoxyuridine (IdU).

All the probes may be labelled with the same tag, but preferably the probes are labelled with at least two different tags, and in a preferred embodiment the probes are labelled with three tags (red, blue and green colors in the case of fluorescent probes).

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescers.

A wide variety of suitable dyes are available, being primarily chosen to provide an intense color with minimal absorption by their surroundings. Illustrative dye types include quinoline dyes, triarylmethane dyes, acridine dyes, alizarine dyes, phthaleins, insect dyes, azo dyes, anthraquinoid dyes, cyanine dyes, phenazathionium dyes, and phenazoxonium dyes.

A wide variety of fluorescers can be employed either alone or, alternatively, in conjunction with quencher molecules. Fluorescers of interest fall into a variety of categories having certain primary functionalities. These primary functionalities include 1- and 2-aminonaphthalene, p,p′-diaminostilbenes, pyrenes, quaternary phenanthridine salts, 9-aminoacridines, p,p′-diaminobenzophenone imines, anthracenes. oxacarbocyanine, marocyanine, 3-aminoequilenin, perylene, bisbenzoxazole, bis-p-oxazolyl benzene, 1,2-benzophenazin, retinol, bis-3-aminopyridinium salts, hellebrigenin, tetracycline, sterophenol, benzimidzaolylphenylamine, 2-oxo-3 -chromen, indole, xanthen, 7-hydroxycoumarin, phenoxazine, salicylate, strophanthidin, porphyrins, triarylmethanes and flavin.

Individual fluorescent compounds which have functionalities for linking or which can be modified to incorporate such functionalities include, e.g., dansyl chloride; fluoresceins such as 3,6-dihydroxy-9-phenylxanthhydrol; rhodamineisothiocyanate; N-phenyl 1-amino-8-sulfonatonaphthalene; N-phenyl 2-amino-6-sulfonatonaphthalene: 4-acetamido-4-isothiocyanato-stilbene-2,2′-disulfonic acid; pyrene-3-sulfonic acid; 2-toluidinonaphthalene-6-sulfonate; N-phenyl, N-methyl 2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine; N,N′-dihexyl oxacarbocyanine; merocyanine, 4(3′pyrenyl)butyrate; d-3-aminodesoxy-equilenin; 12-(9′anthroyl)stearate; 2-methylanthracene; 9-vinylanthracene; 2,2′(vinylene-p-phenylene)bisbenzoxazole; p-bis[2-(4-methyl-5-phenyl-oxazolyl)]benzene; 6-dimethylamino-1,2-benzophenazin; retinol; bis(3′-aminopyridinium) 1,10-decandiyl diiodide; sulfonaphthylhydrazone of hellibrienin; chlorotetracycline; N(7-dimethylamino-4-methyl-2-oxo-3-chromenyl)maleimide; N-[p-(2-benzimidazolyl)-phenyl]maleimide; N-(4-fluoranthyl)maleimide; bis(homovanillic acid); resazarin; 4-chloro-7-nitro-2,1,3benzooxadiazole; merocyanine 540; resorufin; rose bengal; and 2,4-diphenyl-3(2H)-furanone.

In particular fluorescent tags according to the present invention are 1-Chloro-9,10-bis(phenylethynyl)anthracene, 5,12-Bis(phenylethynyl)naphthacene, 9,10-Bis(phenylethynyl)anthracene, Acridine orange, Auramine O, Benzanthrone, Coumarin, 4′,6-Diamidino-2-phenylindole (DAPI), Ethidium bromide, Fluorescein, Green fluorescent protein, Hoechst stain, Indian Yellow, Luciferin, Phycobilin, Phycoerythrin, Rhodamine, Rubrene, Stilbene, TSQ, Texas Red, and Umbelliferone.

Desirably, fluorescers should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Fluorescers are generally preferred because by irradiating a fluorescer with light, one can obtain a plurality of emissions. Thus, a single tag can provide for a plurality of measurable events.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. A diverse number of families of compounds have been found to provide chemiluminescence under a variety of conditions. One family of compounds is 2,3-dihydro-1,-4-phthalazinedione. The most popular compound is luminol, which is the 5-amino compound. Other members of the family include the 5-amino-6,7,8-trimethoxy- and the dimethylamino[ca]benz analog. These compounds can be made to luminesce with alkaline hydrogen peroxide or calcium hypochlorite and base. Another family of compounds is the 2,4,5-triphenylimidazoles, with lophine as the common name for the parent product. Chemiluminescent analogs include para-dimethylamino and -methoxy substituents. Chemiluminescence can also be obtained with oxalates, usually oxalyl active esters, e.g., p-nitrophenyl and a peroxide, e.g., hydrogen peroxide, under basic conditions. Alternatively, luciferins can be used in conjunction with luciferase or lucigenins to provide bioluminescence.

Spin tags are provided by reporter molecules with an unpaired electron spin which can be detected by electron spin resonance (ESR) spectroscopy. Exemplary spin tags include organic free radicals, transitional metal complexes, particularly vanadium, copper, iron, and manganese, and the like. Exemplary spin tags include nitroxide free radicals.

The tag may be added to the probe prior to, or after the hybridization. So called “direct tags” are detectable tags that are directly attached to or incorporated into the probe prior to hybridization. In contrast, so called “indirect tags” are joined to the hybrid duplex after hybridization. Often, the indirect tag is attached to a binding moiety that has been attached to the probe prior to the hybridization. Thus, for example, the probe may be biotinylated before the hybridization. After hybridization, an avidin-conjugated fluorophore will bind the biotin bearing hybrid duplexes providing a tag that is easily detected. For a detailed review of methods of labelling nucleic acids and detecting labelled hybridized nucleic acids, see Laboratory Techniques in Biochemistry and Molecular Biology, Vol. 24: Hybridization With Nucleic Acid Probes, P. Tijssen, ed. Elsevier, N.Y., (1993).

The tag can be attached directly or through a linker moiety. In general, the site of attachment is not limited to any specific position. For example, a tag may be attached to a nucleoside, nucleotide, or analogue thereof at any position that does not interfere with detection or hybridization as desired. For example, certain Label-ON Reagents from Clontech (Palo Alto, California) provide for labelling interspersed throughout the phosphate backbone of an oligonucleotide and for terminal labelling at the 3′ and 5′ ends. As shown for example herein, tags can be attached at positions on the ribose ring or the ribose can be modified and even eliminated as desired. The base moieties of useful labelling reagents can include those that are naturally occurring or modified in a manner that does not interfere with the purpose to which they are put. Modified bases include but are not limited to 7-deaza A and G, 7-deaza-8-aza A and G, and other heterocyclic moieties.

The macromolecule also undergoes “linearization” (before or after binding of the molecular markers on the macromolecules and/or attaching of the tags on the molecular markers), so as to have the macromolecules spread, stretched and extending according to a predetermined direction. In other words, the linearization allows arranging the macromolecules as curvilinear objects. In the present direction, the example of horizontal direction will be arbitrary chosen as said predetermined direction for commodity.

For nucleic acid, in an embodiment, the linearization of the macromolecule is made by molecular combing or Fiber Fish.

Since maximal resolution on combed DNA is 1-4 kb, probes according to present invention are preferably of at least 4 kb.

Molecular combing is done according to published methods (see Lebofsky, R., and Bensimon, A. (2005). DNA replication origin plasticity and perturbed fork progression in human inverted repeats. Mol. Cell. Biol. 25, 6789-6797). Physical characterization of single genomes over large genomic regions is possible with molecular combing technology. An array of combed single DNA molecules is prepared by stretching molecules attached by their extremities to a silanised glass surface with a receding air-water meniscus. By performing fluorescent hybridization on combed DNA, genomic probe position can be directly visualized, providing a means to construct physical maps and for example to detect micro-rearrangements. Single-molecule DNA replication can also be monitored through fluorescent detection of incorporated nucleotide analogues on combed

DNA molecules.

FISH (Fluorescent in situ hybridization) is a cytogenetic technique which can be used to detect and localize DNA sequences on chromosomes. It uses fluorescent probes which bind only to those parts of the chromosome with which they show a high degree of sequence similarity. Fluorescence microscopy can be used to find out where the fluorescent probe bound to the chromosome.

In FISH process, first, a probe is constructed. The probe has to be long enough to hybridize specifically to its target (and not to similar sequences in the genome), but not too large to impede the hybridization process, and it should be tagged directly with fluorophores, with targets for antibodies or with biotin. This can be done in various ways, for example nick translation and PCR using tagged nucleotides. Then, a chromosome preparation is produced. The chromosomes are firmly attached to a substrate, usually glass. After preparation the probe is applied to the chromosome DNA and starts to hybridize. In several wash steps all unhybridized or partially hybridized probes are washed away. If signal amplification is necessary to exceed the detection threshold of the microscope (which depends on many factors such as probe labelling efficiency, the kind of probe and the fluorescent dye), fluorescent tagged antibodies or streptavidin are bound to the tag molecules, thus amplifying the fluorescence. Finally, the sample is embedded in an anti-bleaching agent and observed on a fluorescence microscope.

In fiber FISH, interphase chromosomes are attached to a slide in such a way that they are stretched out in a straight line, rather than being tightly coiled, as in conventional FISH, or adopting a random conformation, as in interphase FISH. This is accomplished by applying mechanical shear along the length of the slide; either to cells which have been fixed to the slide and then lysed, or to a solution of purified DNA. The extended conformation of the chromosomes allows dramatically higher resolution - even down to a few kilobases. However, the preparation of fiber FISH samples, although conceptually simple, is a rather skilled art, meaning only specialized laboratories are able to use it routinely.

An example of protocol of Fiber Fish method is described above:

### Equipment and Reagents

cellular culture

### PBS

### Haemocytometer

lysis solution
5 parts 70 mM NaOH, 2 parts absolute ethanol (Fidlerova et al. 1994). This solution can be stored at RT for several months.

### Method

Take 1-2 ml of cell suspension from the cellular culture.
Wash twice in 5 ml PBS.

### Re-suspend in 1 ml PBS.

Count an aliquot of cells using the haemocytometer.
Dilute cells with additional PBS to give a final concentration of approximately 2×10⁶/ml.
Spread 10 μl of cell suspension over a 1 cm area on the upper part of a clean microscope slide.

### Air dry.

Fit a slide into a plastic Cadenza (Shandon Southern) chamber and clamp in a nearly vertical position.
Apply 150 μl of lysis solution into the top of the cadenza.
As the level drops below the frosted edge of the slide, add 200 μl of ethanol.
Allow to drain briefly.
Holding the edges, carefully lift the slide and cadenza unit out of the clamp.
Pull the top of the slide back from the cadenza, allowing the meniscus to move down the slide.
Air dry at an angle.
Fix in acetone for 10 minutes. Slides can be stored satisfactorily at room temperature for several months.

### Environment

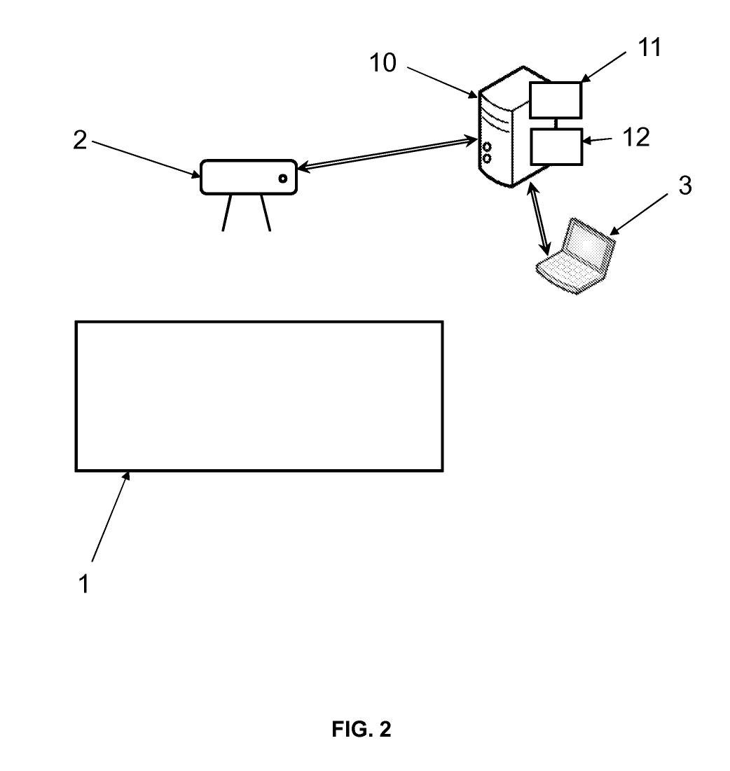

Referring to FIG. 2, the present methods are implemented by a system comprising at least a scanner 2 and equipment 10.

The equipment 10 is typically a server or any computing workstation, and comprises data processing means (a processor 11) and data storage means (a memory 12).

The equipment is connected to the scanner 2, and optionally to a client 3 with a Human-Machine interface for inputting commands, outputting results, etc. The client 3 is typically a terminal such as a PC connected to the equipment 10 through internet, the client 3 implementing a web browser.

The scanner 2 is any sensing device able to acquire at least one sample image depicting said macromolecules (and more precisely the tags attached to) as curvilinear object sensibly extending according to said predetermined direction.

The scanner 2 is in particular an optical sensing device able to sense visible light (and/or non-visible light such as ultraviolet of infrared).

The scanner 2 should be chosen as a function of the type of tags to be detected, as a sample image outputted by such a scanner 2 only represents the tags of the molecular markers. For example, in the case of radioactive tags, the scanner 2 has to be sensitive to ionizing radiations.

According to the present invention, when the labelling is made with fluorescent tags, the reading of signals is made by fluorescent detection: the fluorescently labelled probe is excited by light and the emission of the excitation is then detectable by a photosensor of the scanner 2 such as CCD camera equipped which appropriate emission filters which captures a digital image and allows further data analysis.

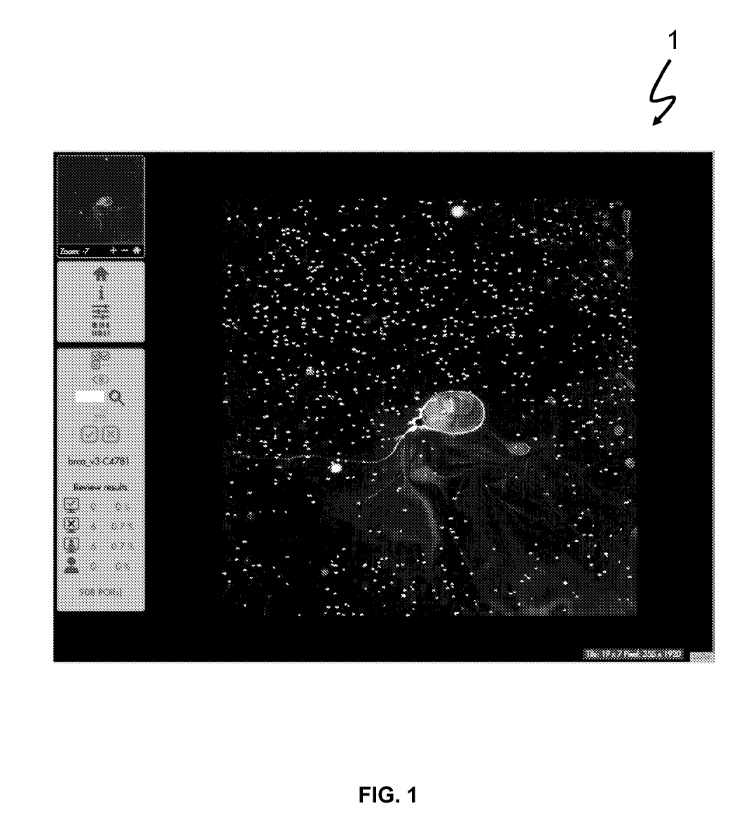

A sample image outputted by such a scanner 2 thus represents red, green and blue spots, see the example of FIG. 1.

To proceed, the user puts one coverslip 1 in the scanner 2, and the latter takes shots of the medium under the coverslip 1.

It has to be noted that the connexion between the scanner 2 and the equipment 10 may be continuous (for example through a network) or intermittent (for example by using memory sticks for transferring one or more sample images).

### First Mechanism—Method for Identification of Target Regions

The present method allows detecting signals in the image, said signals being representations of sequences of tags within the image, i.e. a sequence of target (in other words regions of interest) of the macromolecule (the regions bounds to the molecular markers), in other words code patterns. To this end, will be searched for “candidate” code patterns, according to the following steps.

In a first step (a), the processor 11 of the equipment 10 receives from the scanner at least one sample image depicting the macromolecules, and more precisely presenting said code patterns.

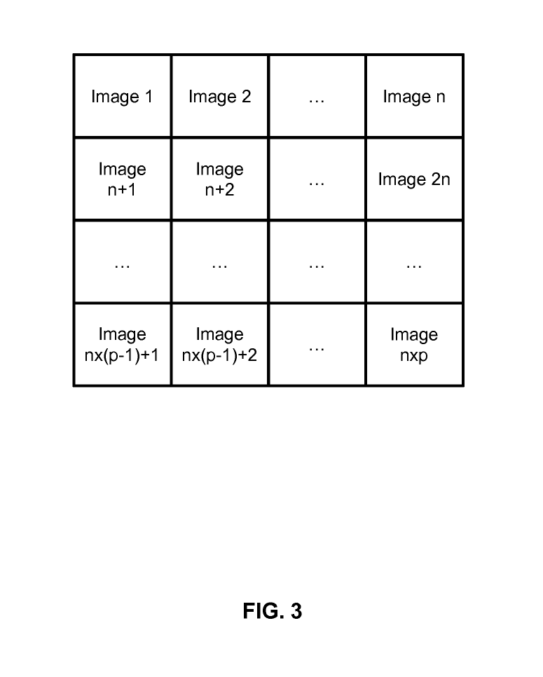

For a given coverslip 1, several sample images are usually generated: the scanner's field of view only covers a small area of the coverslip 1, therefore several fields of view must be scanned in order to cover the whole coverslip 1. These fields of view are then put all together as tiles to make the final image as shown by FIG. 3.

Typical values for n and p are about 50, and more precisely 45 and 42 which makes 1890 fields of view for a whole coverslip 1.

Tiles have typically a size of 2000×2000 pixels, the final image (i.e. the whole coverslip 1) can therefore reach 100.000×100.000 pixels.

Besides, each field of view may be scanned with several fluorophores. Each fluorophore will be associated with a color in the final image. For example, if we use 3 fluorophores (associated with colors red, green, and blue), we will have 3 images per field of view. In case of a plurality of images per field of view, each image is called a channel. In the present description, several images associated with the same field of view (i.e. different colors images) will be treated as independent sample images. It is to be noted that alternatively a single color sample image can be outputted per field of view.

Extra information associated with these images (patient ID, assay type, etc.) may also be received by the processor 11 in the first step (a).

Step (a) advantageously comprises converting the sample images, which are “raw images”, i.e. typically uncompressed and minimally processed 16 bits per pixel per color images. This substep is performed in particular if the images are intended to be visualized by an operator.

In particular, the raw images may be converted into a lighter image format such as jpg, so as to obtain 8 bits per pixel per color images. When converted to 8-bit, each pixel of each image is defined by an integer between 0 and 255.

In a preferred embodiment, for each color (or fluorophore), may be built a single global histogram of pixel intensities from all the raw images or a subset. On each resulting histogram, are computed the min/max intensities so that all pixels with an intensity between min and max correspond to a given percentage (for example 98%) of all pixels of the image. The example of 98% means that once min/max values are computed, all pixels with an intensity below min correspond to 1% of the image, and all pixels with an intensity above max correspond to 1% of the image.

Once min/max values are computed, the intensity of the raw pixels (noted I_{16bits}) is transformed using the following formula:

$${I}_{8 bits} = 255 {( \frac{{I}_{16 bits} - min}{max - min} )}^{1 , 5} .$$

If I_{8bits} is less than 0, it is set to 0, if it is greater than 255, it is set to 255. The power 1.5 has the effect to «shrink» low intensities in order to obtain an image with a darker background.

Any known method can be used to achieve this conversion, in particular local thresholding algorithms (see J. Sauvola and M. Pietaksinen. (2000). Adaptive document image binarization. Pattern Recogn, 225-236) which estimate local adaptative thresholds of image sub-windows.

In a second step (b), the processor 11 of the equipment 10 pre-processes a sample image so as to generate a binary image from the sample image. At least one binary image is generated per field of view (i.e. one for the three samples images corresponding to the three channels of a field of view), and preferably a binary image is generated for each one of the sample images (including different channels of a same field of view, i.e. three binary images are generated for a field of view, said generated binary images being referred to as binary channels).

In an embodiment, a sample image to be pre-processed is thresholded to end up with a 1 bit image.

Several thresholding algorithms could be applied. They are grouped into two categories: global thresholding algorithms (Otsu, N. (1979). A threshold selection method from gray-level histograms. IEEE Trans. Sys., Man., Cyber, 62-66) which estimate a global threshold value, and already discussed local thresholding algorithms which estimate local adaptative thresholds of image sub-windows.

Local approaches are usually more adapted to deal with image artefacts, and are presently preferred.

In a preferred embodiment, is performed an approach that performs local-mean thresholding algorithm while being specific to our images and application requirements, similar to (Rohrer, J. M. (1983). Image thresholding for optical character recognition and other applications requiring character image extraction. IBM J. Res. Dev., 400-411).

Indeed, code patterns to be detected in image are usually with higher intensity than background. Furthermore, because of the linearization they are usually according to the predetermined direction, i.e. horizontal or near-horizontal lines with 10-20 pixels of thickness.

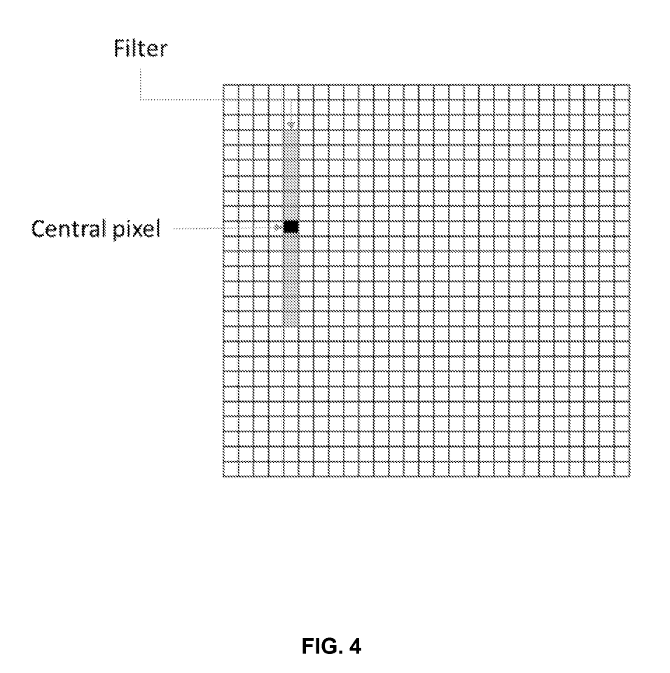

Thus, applying a vertical (i.e. orthogonal to said predetermined direction) local mean thresholding filter, as represented by FIG. 4, of a fixed length, is a good candidate algorithm to better highlighting the useful information (candidate code patterns to be detected).

When applying the filter, the vertical subwindow's threshold intensity is computed. If the central pixel's intensity is higher than this threshold, the pixels takes the Boolean variable 1, otherwise it is set to 0.

The threshold value could be any statistical value related to the subwindow: alpha*mean, alpha*mean+beta*variance, alpha*median, etc.

The “alpha*mean” statistic guarantees the smallest computation time.

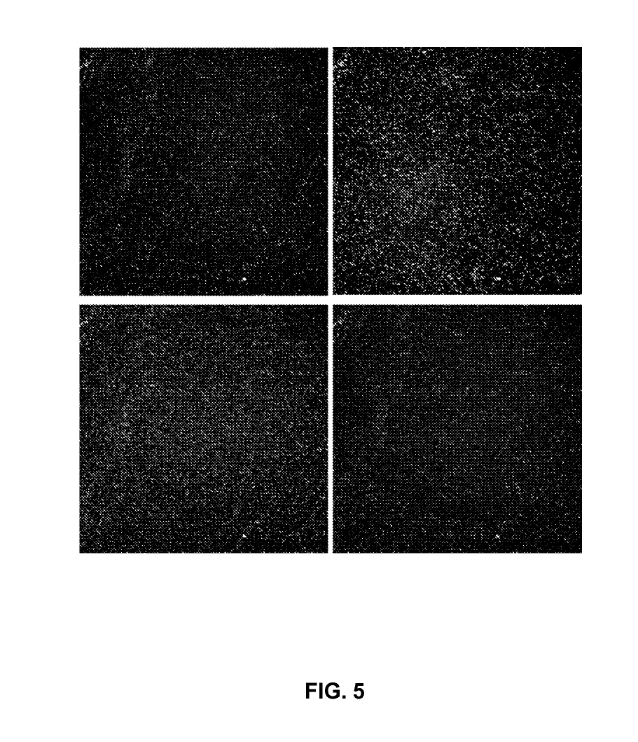

In the represented examples (see for example FIG. 5 which shows the three binary channels from raw images and the initial color image (at the bottom right) as a combination of the three binary channels), 1.2*mean is the chosen threshold, and the size of the vertical filter is 51 pixels.

If a binary image is generated for each channel, the 3 binary channels are preferably fused, so as to obtain a single binary image per field of view.

Merging binary channels could be done with several operations: mean, maximum, etc. In order to keep the maximum of the information contained in these data the “maximum” operation is preferred.

Alternatively, the single binary image for a field of view is directly generated from the different sample images associated to the colors of the field of view.

In an optional step (b′), the generated binary image is post-processed, and in particular “cleaned” so as to remove the unnecessary information.

Indeed, several non-useful objects are usually present in the binary image, as it can be seen in FIG. 1: isolated small spots, large spots or near-vertical curvilinear structures (i.e. structures not extending according to the predetermined direction).

All of these objects, if not removed from the binary image, induce false-positive signals to detect, and increase the computational resources needed for analyzing the image.

In an embodiment, step (b′) comprising the application by the processor 11 of shape based filters to remove such non-useful objects. Object contours in the binary image are first extracted. Shapes are then analyzed by computing some properties: height, width, surface, (height, with) of the smallest rectangle englobing the shape, etc.

Thresholds related to these properties are fixed to remove non-useful objects, as these objects are sensibly larger than the macromolecules of interest (see the “stains” of FIG. 1).

The optimal value of a threshold is computed on reference images. It is optimal if it maximizes the presence of complete true-positive signals, and the absence of other complete parasite object. At the end of step (b′), a filtered binary image is obtained.

In step (c), a code pattern detection is performed. More precisely, for at least one template image, and for each sub-area of the binary image(s) (or preferably of the cleaned binary image(s)) having the same size as the template image, is calculated a correlation score between the sub-area and the template image.

Since the image is binary has no colors nor gray values, the shape of the code pattern is a good property to take into consideration for detecting code patterns.

The present method takes advantage that the shape property to be consider is the curvilinear aspect of the macromolecules depicted. More specifically, a true code pattern should be in most of cases a collection of near-horizontal segments, with quasi-same orientation angle.

The template matching approach in image analysis is thus well suited to this problem, as the code patterns to be detected only present a very limited number of shapes.

This step consists in defining at least on template image that will be searched for inside the binary image.

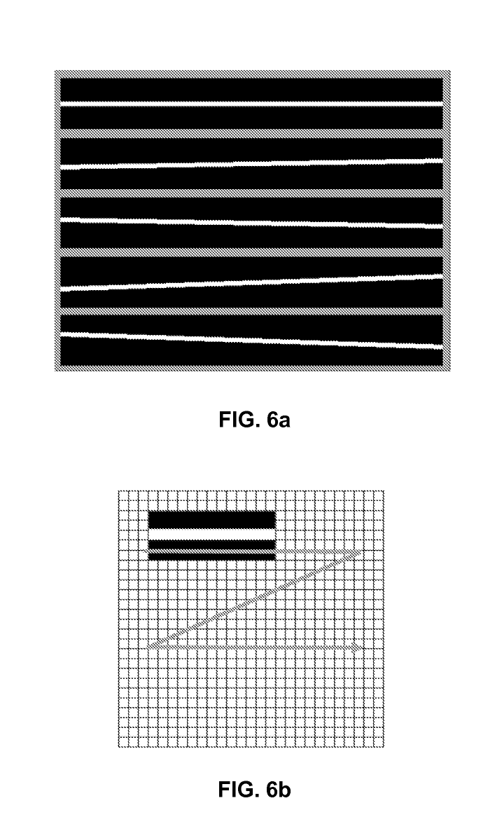

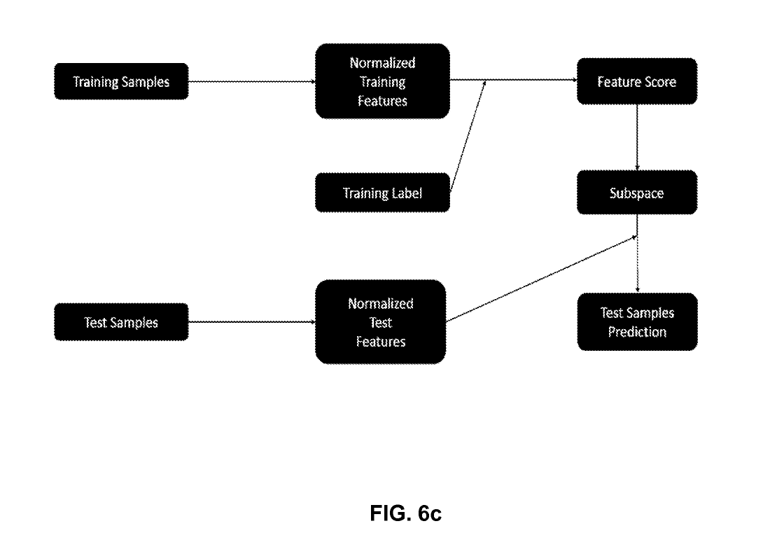

As represented by FIG. 6a, the template images that advantageously corresponds to the requirement is a set of binary segments, in particular a set of oriented binary segments, and preferably the same binary segment oriented according to different directions around the said predetermined orientation of linearization (the horizontal direction in the depicted examples). Preferably, all the template images are rectangles with the same dimensions so as to increase the efficiency.

The size of the segment is for example the maximum size of a true code pattern to detect. The orientations are different small orientation angles from the predetermined direction. The thickness of the segment is fixed empirically.

For the example of BRCA genes (breast cancer), the length of the template segment is 300 kb, the orientation angles are {−6, −4, −2, 0, 2−, 4, 6} degrees from the predetermined direction and the code pattern thickness is 3 kb. The template line is inside an image of shape (300 kb, 10 kb).

The binary image is “scanned” so as to compare each sub-area of the binary image to the template. By sub-area, it is meant a part of the binary image having the same dimensions as the template each. A sub-area may be designated by reference coordinates (in particular x-y coordinates of its centre, or one of its corners).

Preferably, such scanning is performed line by line so as to efficiently wander the whole image, according to the path represented by FIG. 6b. For each sub-area selected, each template image is compared to the sub-area, i.e. a correlation score between the sub-area and the template image is calculated.

By correlation score is meant a score representative of the “similarity” between the two images to be compared according to a given metric. The more the template image and the sub-area are similar, the higher is the score.

Any known similarity metric can be used. For example, the similarity metric may be the “Fast normalized cross-correlation”, or alternatively the score may be simple computed as the number of matching pixels (i.e. pixels having the same value in the sub-area and the template image to be compared) of the sub-area divided by the number of pixels of the sub-area, or the number of matching 1-pixels (i.e. pixels having the same value “1” in the sub-area and the template image to be compared) of the sub-area divided by the number of 1 pixels of the sub-area.

The best locations of the sample image (i.e. the locations which are likely to contain signals of tags of the macromolecules) are the sub-areas with the highest correlation scores. Therefore, a minimum correlation score is fixed to select only the best candidate sub-areas for further inspection. In others words, in a step (d), for each sub-area of the binary image for which the correlation score with a template image is above a first given threshold, the processor 11 selects the corresponding sub-area of the sample image.

When using the normalized correlation as similarity metric, the first threshold is for example fixed to 0.2.

The pre-selection of candidate sub-areas of the sample images drastically reduces the surface of sample images which is ultimately tested, and therefore largely reduces the computation time.

By selecting, it is meant either extracting the sub-area from the binary image and then recoloring it, or only picking the reference coordinates associated with the sub-area, so as to use these coordinates for extracting the corresponding sub-area(s) of the initial sample image(s), and preferably if there are several sample images per field of view (one per color), combining these into a “multi-colored” version of the sub-area of the binary image.

In an embodiment, a candidate sub-area (binary, unicolored, or already multicolored) is modified as a function of the template image with which a correlation has been identified. In particular, this sub-area can be tilted according to an orientation angle associated with the template. For example, if a sub-area of the binary image appears to match with a template image depicting a line with an angle of +X° with respect to the predetermined direction, the candidate sub-area can undergo a tilting of −X° so as to fully extend according to said predetermined direction.

The advantages of this approach are numerous:

Well suited to detect linearized macromolecules because of their sensibly parallel arrangement. In particular, known methods such as the Beamlet transform are generally speaking more efficient, but far less efficient than the present template matching in this specific case;
Robustness (insensitive to anomalies such as mutations, since the mutation has no effect on the linear-structure of the macromolecules and the tags)
Efficiency (Fast-cross correlation method is a very efficient implementation of the template matching algorithm);
Genericity (templates could be adapted regarding the shape of the true code pattern to detect, i.e. length, thickness, continuity, etc.).

In an embodiment wherein there are a plurality of tiles, since steps (b) to (d) are performed in each tile separately, a true code pattern could be shared by two or more tiles, i.e. the representation of a macromolecule of interest may be cut at the junction of two or more tiles. Such a code pattern will be detected as two separate candidate code patterns. A merge operation is required.

Thus, a post-processing step (d′) is advantageously performed in the case of a plurality of images samples associated to different fields of view to improve detection quality.

To this end, candidate code patterns to merge are searched for. Since detection is performed on tiles separately, these code patterns should be in the sample image borders. So, candidate sub-areas at the borders of tiles are first selected. Then, coordinates of these sub-areas are compared to merge pair of ones that are close. The sub-areas suited to the merge operations are replaced by the fused one.

The selected sub-area, the merged ones as well as the individual ones, are then advantageously filtered so as to discard the maximum number of possible false-positive candidates while preserving the possible true-ones.

Indeed, the selected sub-areas may have a truncated or artefactual color sequence. Several technical reasons can explain this:
Partial hybridization of fluorescent probes,
Background fluorescent noise falsely interpreted as informative signal during review,
DNA fragmentation during sample preparation for molecular combing experiments.

The information contained in such sub-area is therefore partial and noisy (labelling errors, underestimation of probe length, are likely), which decreases the resolution of the method.

Filtering will be based on other discriminative properties than the code pattern's shape property, already used in template matching of steps (c) and (d).

For this purpose can be used several filters. Each filter explores a unique property of a true code pattern, called “parameter”. The filter will affect a score to a detected sub-area regarding this parameter. If the score is above a filter's parameter threshold, the sub-area is discarded, otherwise kept as a selected sub-area.

The filter's parameter threshold is fixed using reference sub-areas (set of training examples). Indeed, for a given filter parameter, parameters values are computed on true-positive and true-negative items. An optimal threshold will be the value that separate the two populations, or at least, the value that reduces the overlapping region between the two populations.

A perfect filter is the one that guaranties a good separation between the two populations, or at least that guaranties the smallest overlapping between the two populations.

A bad filter (to no consider) is the one that has a great ambiguity to separate the two populations.

In the represented examples, the parameter of the filter is the number of red, blue, green segments that are above 3 Kb, and a suitable parameter value of the filter is for example 2.

This filtering method could be also solved using machine learning algorithms, in particular according the framework illustrated by FIG. 6c. In this case, filters parameters are considered as “features”. A classifier, such as the SVM, is learned on the training set to discriminate between true and false positives sub-area. Once the classifier is trained, it defines a predictive model used to predict on a given image if the sub-area is a positive signal (to be selected) or a negative one (to be discarded).

To this end, for each item of the training set (both true positive and false positive sub-areas with labels ‘validated’ and ‘discarded’ respectively for which depicted target regions have been successfully identified in the step (f) that will be explained below) the set of said features are extracted in order to train a machine learning discriminative model.

These features are for example chosen so as to characterize the noise, the linearity of the signal, the characteristics of connex components detection in the image, the rate of mixed colors (for separating relevant signals from mixed fibers, etc.), examples thereof will be detailed below. No information about particular chaining of colored probes, i.e. code pattern, is yet to be included, in order to avoid discarding mutated signals, because potential genome modifications may contain different colors chaining. This is of primary importance given the very small number of mutated signals present in the training databases.

The use of features is very advantageous, because their extractions are not time-consuming. The computation time should indeed not be superior to a quarter of second per region of interest in order for the computation of the filter on slides (that can contain more than 2500 candidates signals) to be under a dozen of minutes.

To the contrary, as it is necessary to extract a maximum of various information in order for the machine learning filter to be robust and efficient, alternative methods such as segmentation methods (level-sets or graph-cuts) are not possible. Similarly, methods as deep-learning for segmentation cannot be considered because of the cost of precise labeling of signals contours and because of the very important number of signals necessary to model the complexity of the task when avoiding overfitting issues, that are hard to deal with when using these methods and this, even with the use of data augmentation.

A list of preferred “features” for the machine learning will now be presented. The skilled person can use one or any number of them. They allow characterizing an important number of relevant information for classifying true positive

DNA signals from false positive ones. Several features aim at extracting the same information in different ways in order to increase the robustness to potential estimation errors caused by the presence of noise and the variability in images:

Intensity distribution. For extracting this feature, the RGB image of the candidate sub-area is transformed towards its Hue-Saturation-Value (HSV) representation. A mask of the Value image is computed on the parts where Value is inferior to some threshold as well as on the parts where Saturation is inferior to some threshold. This way, sub-areas of intensity that is too small for belonging to a potential signal are discarded as well as mixed fibers, which frequently appear in white and are thus under-saturated. The obtained gray level image allows characterizing the global distribution of the values of this image according to the following procedure:

Resizing of the image for size reduction,

Vectorization of the obtained image, and sorting of this vector,

Adding to the feature set the mean, standard-deviation, and a re-sampling of this vector on some fixed number of points,

Computation of the derivative of this vector,

Extracting as feature the mean, standard-deviation, and a re-sampling of this derivative on some fixed number of points.

Vertical mix of colors, which are frequent when DNA fibers are mixed. For extracting this feature the HSV representation of image is used and a filtering based on Hue is performed for obtaining binary images corresponding to parts of different colors (Blue, Red, Green, Yellow, Magenta and Cyan). For each of these images a convolution with a vertical filter is computed. Then, the binary image where activations correspond to areas where at least two convolution output images are positives (two distinct colors that are vertically close) is computed. The obtained image may be finally characterized the same way as for the intensity distribution.
Candidate signal linearity. For extracting this feature, a Radon transform computed on a set of angles close to horizontal may be used. For each of these angles, are obtained vectors whose points correspond to the different vertical offsets of the lines. A convolution of these obtained vectors with a filter whose size is close to the mean width of molecular combing DNA signals is performed, then a maximum operation on the results on those convolutions (the maximum value being the extracted feature).
Rotation and cropping to zoom in on the potential signal. An estimate of the angle is obtained by using the angle where the maximum of the Radon transform occurs, and an estimate of the vertical offset of the line by using the one where the maximum of the Radon transform occurs. A rotation of this angle on the opposite direction is performed and an horizontal stripe centered at the corresponding offset and of some fixed width is extracted. The horizontal profile of this stripe is computed by summing the intensities with respect to columns. The centroid of this profile is computed to estimate the center C of the signal according to the horizontal axis. Then, a set of windows centered on C and of increasing lengths is defined. For each window, the ratio of the sum of the profile elements within the window divided by the total sum of the profile elements can be computed. By considering the window size from which the ratio is over some threshold, an estimate of the signal size can be obtained, so as to crop the candidate signal.
Horizontal color changes, because mixed fibers can contain very small color probes changing horizontally at a fast frequency while color probes of true signals are larger and thus evolve horizontally in a slower way. Horizontal profiles are computed for the three color channels R, G, B by summing the sub-image with respect to the columns. For each profile, and for a set of thresholds, the profile is binarized and its derivative is computed, so as to calculate the mean of those derivatives, which is extracted as a feature (linked to the number of horizontal color changes by length unit).
Connex components shapes. To characterize these shapes, is first computed a binarization of the image Value of the HSV representation of the sub-image. Connex components in this image are extracted so as to compute their areas and eccentricities. The mean of the eccentricities of the components whose areas are superior to some threshold as well as the mean of the areas of all connex components are extracted as features.
Thick parts. A convolution with a vertical filter of a binarization of the gray level image obtained when characterizing intensity distribution is performed. Then, the number of pixels for whose the convolution output is superior to some threshold divided by the total number of pixels of the image is extracted as a feature. This allows estimating the rate of areas whose thickness is superior to some number of pixels, which can be extracted as a feature.
Parts of homogeneous colors. The set of pixels that are superior to some threshold as points in a space whose features are the coordinates of the pixel as well as the intensity values of each R, G, B channels. Is considered for performing a k-means clustering on those points to obtain the connex parts homogeneous in colors. The standard deviation of the intensity values R, G, B is compute for the different obtained parts to extract information about the color homogeneity inside the parts. We add to the feature set the mean and standard deviation of the obtained homogeneity values on the set of clusters.
Aligned components. A RANSAC-based approach is used on the centroids of the connex parts homogeneous in colors for computing the subset of areas whose centroids are the most aligned. The number of obtained components, the quadratic alignment error of the selected components, the mean of the areas of connex components, as well as the mean of the eccentricities and color homogeneities can be extracted as features.
Linearity of the candidate signal after denoising. The linearity of the signal may also be characterized using a Radon transform after a step of denoising. A proposed denoising/binarization method works as follows:

For each RGB channel,
binarizing the image for only keeping 10% of the highest intensity pixels,
performing a minimum filtering with an horizontal filter for only keeping the parts that are sufficiently spread horizontally,

extracting the connex components and filtering them for keeping only those with an area superior to some threshold, with eccentricity superior to some threshold and that are oriented close to the horizontal line,

performing a XOR operation of the obtained binary images for getting rid of areas of mixed colors.

Saturation distribution. May be added to the set of features a histogram of the Saturation image values from the HSV representation of the initial image.
Co-occurrence matrix, for characterizing globally the image texture using information about co-occurrences of gray-levels. The Value image is quantified on 3 bits for defining 8 level of grays. Then, the sum of the pairs of pixels having some difference in terms of gray-levels and located with a particular distance and orientation from each other is computed. Several square matrices of size the number of gray-levels are obtained. The number of matrices is the number of considered distances times the number of considered orientations. Using these matrices, several information can be extracted as features: contrast, dissimilarity, homogeneity, energy and correlation of gray levels with respect to distances and orientation.

When extracting the features for the N items of the training set for learning the predictive model, are obtained a set of N vectors of K features and a vector of N labels corresponding to the state 0 (rejected region of interest, false positive), or the state 1 (accepted region of interest, true positive).

A feature can be considered relevant for the prediction of a label if the knowledge of the feature gives information on the label. The functional relationship between a feature and a label can be non-linear. Local variances of the label with respect to each of the features could be computed for estimating a score close to conditional entropy as follows:

Getting index corresponding to the sorting of the features,
Re-arranging the label with respect to those index,
Computing, on a plurality of sub-windows without overlap the variance of the labels on each window,
Summing those variances.

The smaller the score is, the more the corresponding feature brings information for prediction the label. Is then selected a set of k<K features using these scores. Finally, the training samples are embedded within the space of those selected features with a weighting of the feature using the inverse of the corresponding score. The more important the feature is, the more the corresponding axis will have impact on the relative location of the points.

Before the step of feature weighting, can be performed perform a standardization of those features to bring their mean towards 0 and their standard-deviation towards 1 on the set of training regions of interest in order to have an equal contribution of all features in the definition of the subspace before weighting.

Finally, the predictive model is defined for example by a non-parametric gaussian Nadaraya-Watson kernel regression in the obtained subspace.

In such a case, one hyper-parameter needs to be estimated (the spread of the gaussian used for kernel computation). This parameter can be optimized on the learning database using a slide-specific 6-folds cross-validation. Machine learning is suited to solve the filtering task when more than two filters are necessary. Otherwise, the previous approach, which is a rule based one, is easiest for design and interpreting the filters properties.

Since the shape property is not the discriminative property of a true code pattern to detect, the selected sub-areas are only candidates (false-positive code pattern are detected by the template matching of steps (c) and (d) in addition to the true-positive ones).

Therefore, is performed by the processor 11 a step (e) of calculating, for at least one reference code pattern, and for each selected sub-area of the sample image, an alignment score between the sub-area and the reference code pattern.

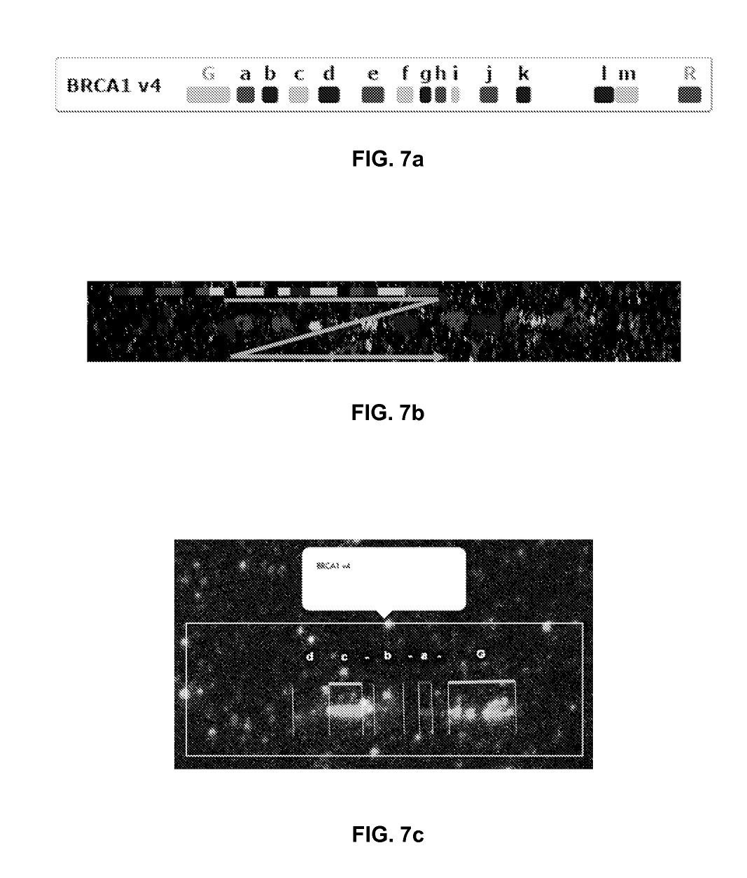

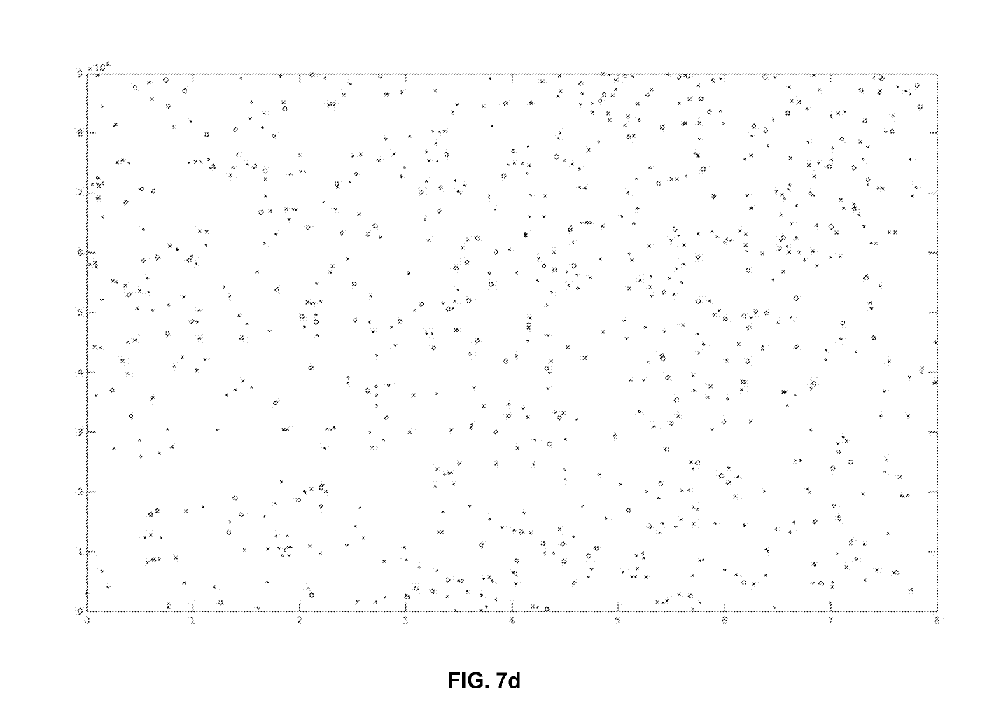

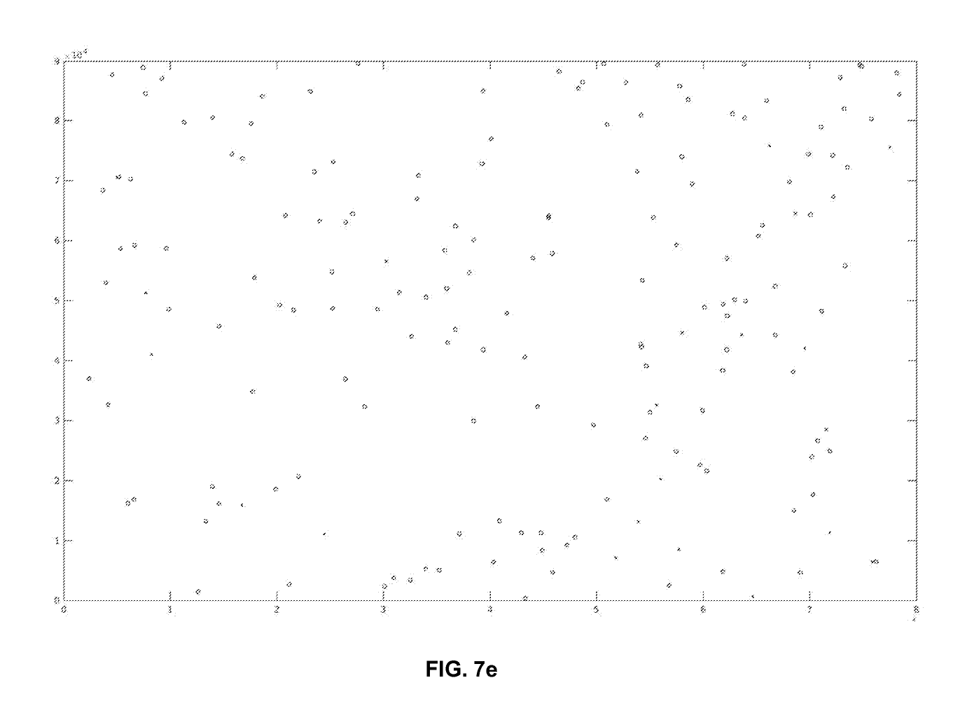

Such step (e) is somehow similar to step (c) of pattern matching, except that said reference code pattern is not an image, but is defined by a given sequence of tags such as represented by the example of FIG. 7a (still BRCA1 gene).

In a step (f) somehow similar to step (d), for each selected sub-area of the sample image for which the alignment score with a reference code pattern is above a second given threshold, each target region depicted in said selected sub-area is identified among the target regions associated with the tags defining said reference code pattern.

More particularly, each reference code pattern is the true code pattern of a reference spatial organization of a fragment of said macromolecule, i.e. a gene type in the case of a nucleic acid (without anomaly), and is characterized by:

a type of tag (i.e. a color for fluorescent tags);
a length of the tag (representing a length of the labelled marker, express in kb for the probes of DNA);
a mark identifying the target region associated with the tag among others within the code pattern (a letter in the example of FIG. 7a);
when required, position and width of gaps between the tags.

Any selected sub-area of the sample image (if confirmed as a true-positive) also defines a candidate code pattern as a sequence of tags, and has to be classified into one of the reference code patterns, aligned along the right code pattern and each tag (each colored segment) in the sub-area has to be assigned to one of the molecular makers of the associated reference macromolecule.

The discriminative property between reference code patterns is the color-length sequence. So classifying and labelling a selected sub-area should consider this property in order to decide to which reference code patterns the sub-area is more similar and the location of each tag.

Color-length similarity between the candidate code pattern of a sub-area and the reference ones is computed using a sequence-matching approach.

As already explained, state-of-the-art matching algorithms are divided into two classes: global and local approaches. Global sequence matching approaches try to find a global alignment between two sequences, while local approaches check the alignment locally.

The present method proposes a new matching approach that globally aligns the sequences in a first sub-step, then a local refinement technique is applied to improve the labelling quality. For example, the global alignment sub-step is based on a correlation matching algorithm. Other methods could be implemented as well (such as Needleman & Wunch, as defined in Needleman, S. B., & and Wunsch, C. D. (1970). A general method applicable to the search for similarities in the amino acid sequence of two proteins. Journal of Molecular Biology, 443-53, and Smith & Waterman, as defined in Smith, T. F., & and Waterman, M. S. (1981). Identification of Common Molecular Subsequences. Journal of Molecular Biology, 195-197.). Each reference code pattern is moved along the candidate code pattern of the sub-area. At each position, a correlation metric is computed between the overlapping parts of the two code patterns to compare (see FIG. 7b). The position that gives the best correlation score is considered as the best global alignment with that reference code pattern, i.e. with the highest alignment score.

The class of the reference code pattern giving the best global alignment score is affected to the candidate code pattern.

Candidate patterns in the image are usually with different color-length sequence than the theoretical one. The main reasons are the following:

Stretching factor: this is a consequence of the linearization. Candidates patterns are stretched according to different stretching factors (for example between 70% and 130%) at the end of the operation, ending up with a plurality of candidate code patterns with different lengths for the same sub-area, compared to the reference one. The stretching factor could be code pattern-dependent. For some complex rare cases, it could be molecular marker-dependent.

Orientation: the linearization makes the macromolecules all extending sensibly according to the predetermined direction. However, for this direction there are two opposite orientation which are possible. For example, horizontal macromolecules can be read either from left to right or from right to left. Therefore candidates patterns are mirrored as to provide for each one (for each stretching factor) the symmetric candidate code-pattern, compared to the reference one.

Mutation: Abnormal macromolecules will present different color-length-ordered sequences compared to the reference one. Thus, candidate code patterns would have different sizes (globally, or inside some tags) and also different rearrangement of regions.

Hence, a global alignment is not always sufficient for regions identification.

A local alignment step is performed to adjust locally the tag locations. The algorithm used is based on replacing non-matched regions of the candidate code pattern by the neighboring ones. If a neighbor region, with a same color, exists, the non-matched region will be associated its tag. Otherwise, the color of the region is considered as the associated mark (instead of being marked “a”, “b”, “c”, etc., the region is marked “RED”, “GREEN”, etc.) . Regions labelled with color names marks are considered as ambiguous regions, where a potential mutation is happening, as it will be explained later.

### Outputting & Manual Review

In a step (g), the processor outputs (preferably to the client 3), the different target region(s) identified. As hundreds of copies of the same macromolecules are generally present in the same coverslip 1, the same sequence of regions is identified numerous times, and only a few different sequences of target regions are identified.

Therefore, preferably, only the distinct sequences of target regions are outputted, in particular along with their occurrence rate. The output can include the selected sub-area of the sample image, on which is represented the sequence of identified target regions (see the example of FIG. 7c).

Optionally, in a step (h), the equipment 10 receives validation data from an operator using the client 3.

More precisely, an operator may proceed to manual review, by controlling and correcting (when necessary) the results of detection and classification algorithms presented above. More particularly, an operator may be asked to

Discard candidate code patterns that do not correspond to regions of interest;
Control and possibly modify the beginning and end of each candidate code pattern;
Control and possibly modify the classification of the candidate pattern (i.e. the reference code pattern selected);
Control and possibly modify the tags attributed to each measurement (marks of probes when the measurement can be matched to a target region, color name otherwise).

### Method of Machine Learning

According to a second aspect is proposed the standalone method of machine learning preferably performed at step (d).

As explained, this method proposes to learn a model used for predicting the status of the test candidate's signals, to extract features for each sub-area, to select relevant features, and to predict class of an object (with a ranking weight) using the predictive model trained previously during a machine learning phase.

Such method may be performed on any sub-areas of any image directly provided to the processor. Consequently, the images can be provided by any scanning machine such as manual or automatic digital scanners with fluorescence or any other modality.

Such method of identifying at least one sequence of target regions on a plurality of macromolecules to test, each target region being associated with a tag and said macromolecules having underwent linearization according to a predetermined direction, comprises performing by a processor 11 of equipment 10 the following steps:

(α) receiving a plurality of candidate sub-areas of a sample image from a scanner (2) being sensitive to said tags, each sub-area possibly depicting one of said macromolecules as a curvilinear objects sensibly extending according to a predetermined direction;

(β) applying on the candidate sub-areas a thresholding filter using machine learning algorithms so as to select relevant sub-areas (preferably according to embodiment described for the method according to the first aspect);

(χ) For at least one reference code pattern, and for each selected sub-area, calculating an alignment score between the sub-area and the reference code pattern, said reference code pattern being defined by a given sequence of tags;

(δ) For each selected sub-area of the sample image for which the alignment score with a reference code pattern is above a second given threshold, identifying each target region depicted in said selected sub-area among the target regions associated with the tags defining said reference code pattern;

(ϵ) Outputting the different sequence(s) of identified target regions.

Steps (γ, δ, ϵ) are advantageously similar to steps (e, f, g) of the method according to the first aspect.

The skilled person will know how to adapt any embodiment of the method according to the first aspect to this second aspect method.

### Results

The applicant has performed test on the BRCA genes so as to compare the quality of the present method. For three tests, the efficiency and the purity of the results have been calculated when using the known Beamlet transform method, and when using the present method.

The efficiency, also known as the sensitivity, measures the proportion of positives that are correctly identified as such, and is computed as the following:

$$Efficiency = \frac{True positives}{True positives + False negatives}$$

The purity, also known as the precision, measures the accuracy of the system, and is computed as the following:

$$Purity = \frac{True positives}{True positives + False negatives}$$

True positives are the correctly identified true sub-areas, False positives are the incorrectly identified true sub-areas and False negatives are the incorrectly rejected (or undetected) true sub-areas.

In the first case (Beamlet transform method), the efficacy ranges from 32% to 43%, and the purity ranges from 27% to 53%.

In the second case (new identification method), the efficiency now ranges from 60% to 83%, and the purity ranges from 54% to 74%. If a filtering step based on a predictive model defined by machine learning is further performed at step (d), the efficiency even ranges from 67% to 96%, and the purity ranges from 62% to 98%.

Therefore, the efficiency has been doubled while the purity has improved in every test.

FIGS. 7d and 7e demonstrates more visually the efficiency of a filtering step based on a predictive model defined by machine learning (o—true positive signals, x—false positive signals). In this example the machine learning approach improves detection of sequences related to Facioscapulohumeral Muscular Dystrophy disease. FIG. 7d shows many false positive signals detected by the system without machine learning approach and thereafter discarded by the user. Applying the machine learning method select true positive objects and a very few false positive signals demonstrated in FIG. 7e that improve significantly performances of the system.

### Second Mechanism—Method for Analyzing a Sequence of Target Regions

The present method allows performing statistical analysis on code patterns identified in the image, so as to detect anomalies within the macromolecules, i.e. “statistically significant non canonical events”.

Biologically speaking, in the embodiment wherein the macromolecules are nucleic acids, anomalies are large rearrangements in a set of genes of a size range that is compatible with molecular combing technology (of the scale of about 10-100 kb). The assumption made on biological a priori is that there is no more than one rearrangement per DNA on one of the tested genes and that the rearrangement, when present, is appearing on all copies of one of the two alleles of the mutated gene. In other words, the assumption is made that two population are presents, the first (representative of a first allele on a first strand of DNA) being “normal”, and the second (representative of a first allele on a first strand of DNA) presenting the anomaly. No mosaicism (i.e. two or more populations of cells with different genotypes in one individual) is assumed to occur.

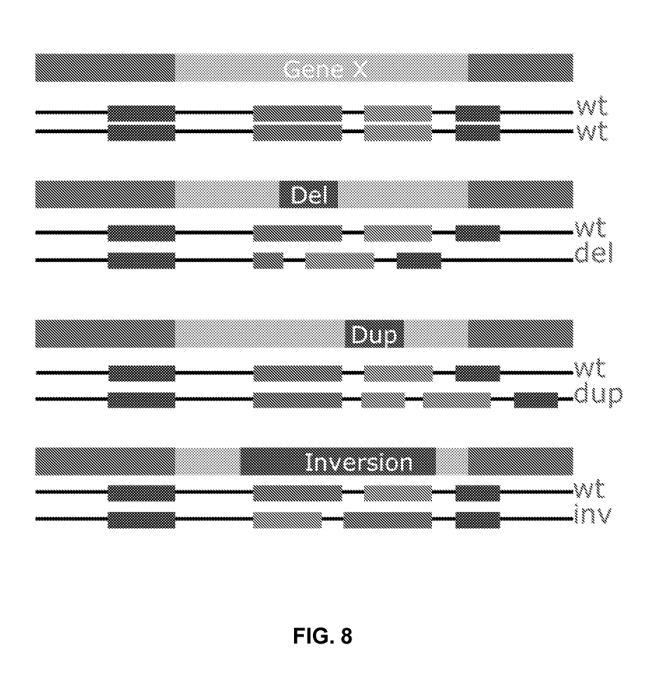

Several types of anomaly are presently detectable: deletions, insertions, duplications, inversions or translocations (see FIG. 8 for examples).

The present method starts with a step (a) of identifying said sequences of target regions from at least one sample image received from a scanner (2), said sample image depicting said macromolecules as curvilinear objects sensibly extending according to said predetermined direction.

Said step (a) is advantageously performed according to the method of the first mechanism (possibly without the outputting step (g)). Alternatively any known identification method such as Beamlet transform method, even if the method of identification as previously disclosed is preferred for efficiency and quality of results. As this point, a code pattern of each sequence is available, such as the one of FIG. 7c. Such code pattern may not exactly correspond to a reference code pattern, in particular if there is an anomaly. The present method will assess if there is effectively an anomaly, or only an artifact, a measurement problem, a defect of samples, etc.

As it will be explained, the present analyzing method relies on the detection of two phenomena, bimodality, breakpoint occurrences, which are likely to be caused by anomalies of the macromolecules, and which will be explained below.

The present method indeed resumes the search for any type of large rearrangements as a search for two distinct populations in target region length distributions (i.e. detection of bimodalities) and a search for favored positions of cut (i.e. detection of breakpoints).

Step (a) advantageously comprises a further sub-step of gap labelling. Indeed, as already explained the target regions are advantageously labelled with marks such as letter in the initial identification method, but not the gaps between the target regions (i.e. the regions without tags, i.e. the non-colored spaces).

For complete anomaly detection, it is preferred to identify the gaps that correspond to theoretical gaps, which may be in a similar way to tags characterized by:

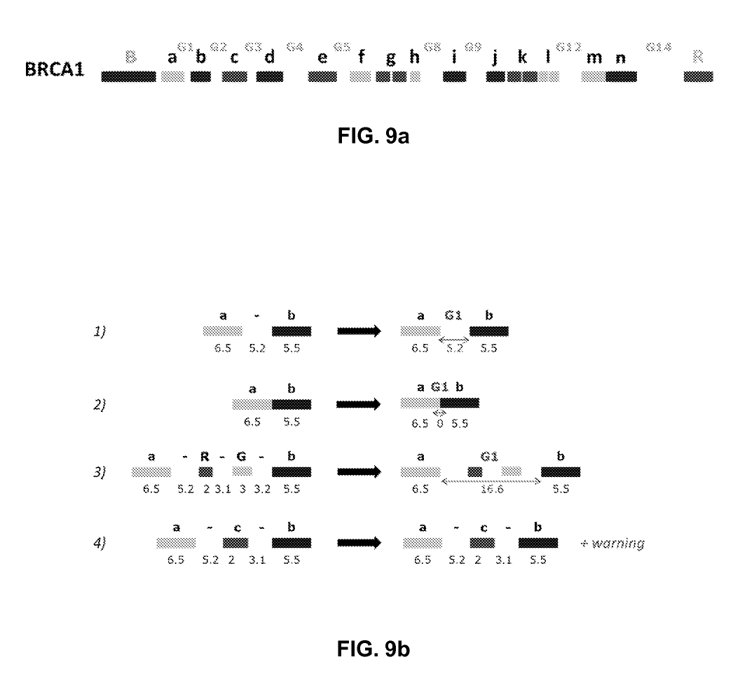

a length of the gap (representing a distance between the closest neighbour regions, express in kb for the probes of DNA);
a mark identifying the target region associated with the gap among others within the code pattern (a mark such as “G1”, “G2”, etc. in the example of FIG. 9a which depicts the example of FIG. 7a with labelled gaps, only marks of gaps with a length over 2 kb being shown);

The gap mark attribution is advantageously performed as follows.

Firstly, is determined the biological direction of the code pattern, either forward or backward (defined as the direction in which the maximum number of target regions is rightly ordered). For example, the code pattern of FIG. 7c is backward. The algorithm returns a warning when a direction cannot be determined.

Then, for each couple of theoretical consecutive regions (such as “a & b”, “b & c”, etc.) with “consecutive” marks (in the sense that there is no other labelled region between them, except ambiguous regions only labelled with their color, no mark), are gathered all measurements in between (gap or color label) as one and attributed the correct theoretical gap mark. In case there is no measurement in between, a measurement of 0 kb is introduced. FIG. 9b represents examples of all the possible cases.

This step of gap labelling also enables to detect errors of target region attribution during manual review. Indeed, inversions in their order (detected when measurements of theoretically consecutive regions are separated by another region with a mark) are notified in warnings returned by the algorithm.

As already discussed, the macromolecules may have been stretched during the linearization. However, there is no guarantee that the stretching factor values of different experiments or datasets are identical. Thus, step (a) advantageously comprises a normalization sub-step for correct length measurements analysis (required for the bimodality detection) and merging of different code patterns datasets.

For each sequence of the set, the processor 11 calculates to this end a global stretching factor value and applies a normalization factor such that this value becomes a normalized one, in particular the value 2. All lengths of target regions of the sequence are corrected using this normalization factor.

In an embodiment, the global stretching factor value is computed as the median of stretching factor values for each code pattern.

Are preferably not considered the first and last regions (as their length measure cannot be trusted)

The length of the sequence is determined as the sum of lengths of target regions and gaps of the sequence, and compared with a theoretical length (sum of the theoretical lengths of the regions and gaps).

$$SF = 2 * \frac{{S}_{theoretical}}{{S}_{measured}}$$

In an embodiment, an iterative process between normalization and anomaly detection is introduced, such that sequences detected as abnormal are excluded from estimation of global stretching factor value, until convergence on normalization factor value and anomaly detection results.

Once the set has been corrected and normalized, the processor 11 looks for anomalies and performs steps (b) and (c) respectively of bimodality detection and breakpoint detection (these steps can be switched).

To detect bimodality, is made the assumption that region length is independent from one target region to another. Thus is subdivided the problem of finding bimodality on a multivariate dataset with missing data (due to cuts of the macromolecules of an identified code pattern) into independent subproblems of finding bimodality for each region or gap of the reference code pattern.

It is to be noted that alternatively the independence assumption between regions may be dropped and bimodality analysis be performed on multivariate data.

When making the independence assumption, step (b) preferably consists in determining if there is at least one target region presenting a bimodal distribution of lengths of said target region. Preferably, the detection of bimodal distribution may be a function of a kurtosis value of the lengths of said target region, or of similar parameters (such as the dip test of Unimodality or EM models, as defined in The Dip Test of Unimodality The Annals of Statistics, Vol. 13, No. 1. (1985), pp. 70-84 by J. A. Hartigan, P. M. Hartigan and the methods described in Hellwig B., et al. (2010). Comparison of scores for bimodality of gene expression distributions and genome-wide evaluation of the prognostic relevance of high-scoring genes. BMC Bioinformatics, 11: 276.).

More precisely, for each target region of said sequences, is calculated the kurtosis value of the lengths of said target region, and said target region being determined as presenting a bimodal distribution of length only if said kurtosis is below a given threshold.

Based on preliminary study on simulated data reproducing the dataset size and variability commonly encountered in molecular combing experiments, such solution using the kurtosis value was proven to provide the best results, when compared with similar methods as cited above.

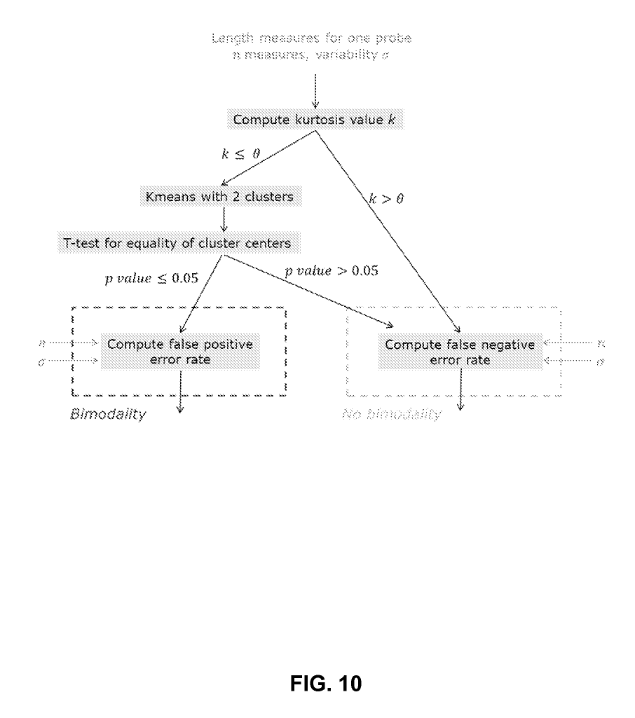

Consequently, is used said threshold 0 on the kurtosis value to distinguish between unimodal and bimodal distributions, as described in FIG. 10.

For example, value around −0.924 could be chosen as the threshold θ. Such values appear to be effective from simulations of univariate Gaussian data reproducing different experimental conditions such as:

Various number of measurements;
Various measurement variability;
Various difference lengths between normal and abnormal measurements (i.e., various deletion or duplication sizes)

Statistical tables of false positive and false negative error rates have been computed from these simulations, depending on the number of measurements and standard deviation of the data.

The threshold value above computed minimizes the false positive and negative error rates over all experimental conditions, with 500 simulated datasets per condition.

In case of suspected bimodality, are advantageously identified two populations of the set of sequences according so the length of said target region (called clusters of length measurements). In an embodiment, the k-means algorithm is used to these different clusters.

It enables to classify sequences with “normal” length measurements (i.e. belonging to the cluster with values closer to theoretical length) and sequences with “abnormal” length (all measurements belonging to the remaining cluster).

Then a t-test is preferably performed so as to verify that the two population have statistically different means.

This t-test is performed on the equality of means of the two clusters, and is verified if its calculated p-value is below 0.05 (see FIG. 10).

When the bimodality has been validated, a false positive error rate may be read from a reference statistical table which takes the number of measurements n and variability a as entries.

When, indeed, no bimodality has been detected or some was detected but not confirmed by t-test, a false negative error rate is read from a reference statistical table which also takes n and a as entries.

Another confirmation step of bimodality detection may be computed, based on error rate values.

In an embodiment, sensitivity analysis is computed on kurtosis values in order to improve robustness to outliers.

In step (c), (which can be performed before step (b)), the processor 11 determines if there is at least one recurrent breakpoint position in said sequences of target regions.

A breakpoint corresponds to a favored position of cut of the macromolecule along a code pattern.

In order to detect such breakpoints, step (c) advantageously comprises estimating rates of sequences of the set being cut at different positions along the code pattern. The position of a cut is defined by the regions between which the cut occurs. For example, the sequence of FIG. 7c stops at region with mark “d”, i.e. the cut is between regions “d” and “e”, and is designated “d ⊕ e”.

Each cut rate is function of the number of sequences comprising both surrounding regions (i.e. without cut, for example “d & e”) divided by the number of sequences containing at least one of the surrounding regions (i.e. with or without a cut, for example “d | e”).

$${c}_{d \text{-} e} = ( 1 - \frac{\sum {I}_{d \& e}}{\sum {I}_{d \vert e}} )$$

For example, if there are 3 occurrences of “d” and/or “e” but only 2 of “d” and “e”, then the associated cut rate is 33%.

A breakpoint is determined recurrent if its cut rate is above a threshold.

Such thresholds for detection of abnormally high cut rates can be determined using simulated data for each breakpoint position.

In an embodiment, are defined a reference dataset of experimental data for which are computed reference cut rate intervals for each breakpoint position.

Then are computed a large number (several thousands) of simulated cut rates from binomial distributions with probabilities within reference cut rate intervals and number of trials mimicking various dataset sizes. Statistical tables of false positive and false negative error rates have been computed from these simulations, depending on the number of measurements and breakpoint positions along the code pattern.

“Abnormal” cut rates are computed in the same way but with different cut rate intervals (approximately the double).

Threshold values can thus be chosen as the ones minimizing false positive and false negative error rates of detecting abnormally high cut rates.

It is to be noted that the threshold values depend on the position of the breakpoint along the code pattern and on the experimental protocol of linearization (especially the DNA extraction step in the case of combing, which impacts the size distribution of code patterns). Consequently, a set of threshold values for breakpoint detection is specific to a particular experimental protocol and has to be recomputed each time the protocol is modified.

In the case where several recurrent breakpoint positions are determined, the false positive error rate computed is the sum of all false positive error rates for each breakpoint position.

If at least one target region presenting a bimodal distribution of length and/or at least one recurrent breakpoint position has been determined, the set of sequences of target regions as being is classified in a step (d) as being abnormal.

In this step (d), the type of anomaly is advantageously identified.

In particular:

the detection of a breakpoint is a good indicator for the presence of an inversion or a translocation;
the detection of more than one breakpoint is a good indicator for the presence of a deletion of entire region(s) of the code pattern;
the detection of bimodality is a good indicator for the presence of a duplication or deletion in the regions;

In the case where no anomaly is detected (neither bimodality nor breakpoint), a resolution for anomaly detection on each region may be computed, based on false negative rates of bimodality and breakpoint detections, mentioned before. This resolution value depends on the quality of the data, i.e., the number and variability of length measurements. Resolution values for regions of a code pattern are computed by taking the maximum value of all resolutions of the probes in these regions.

Step (d) comprises outputting the results of the anomaly identification, in particular through the client 3.

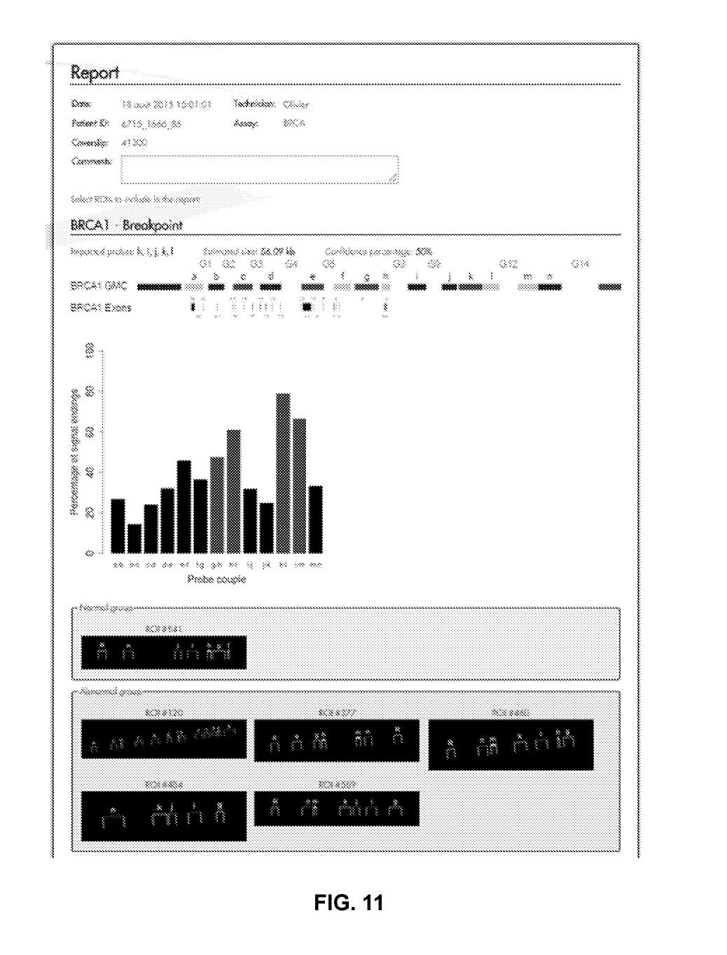

In a preferred embodiment, is output is report such as represented by FIG. 11 (still the example of BRCA gene).

This report may comprise:

A list of the phenomena detected (Bimodality, breakpoint or no anomaly)
for each phenomenon detected:

Characterization of the detection (regions impacted, estimated length of anomaly);

The confidence percentage of the detection (or resolution when no anomaly is detected);

All values used for generating report graphics
Code patterns of signals of normal and abnormal groups

### Equipment

In a third aspect, the invention relates to the equipment 10 for implementing the method of identifying at least one sequence of target regions on a plurality of macromolecules to test according to any aspect of the first mechanism and/or the method of analyzing a set of sequences of target regions on a plurality of macromolecules to test so as to detect anomalies therein according to the second mechanism.

As already discussed, the equipment 10 is typically a server, comprising a processor 11 and if required a memory 12. The equipment 10 is connected (directly or indirectly to a scanner 2).

The present invention also relates to the assembly (system) of the equipment 10 and scanner 2, and optionally at least one client 3.

If configured for the first mechanism, the processor 11 implements:

A module for receiving from the scanner 2 connected to said equipment 10 at least one sample image depicting macromolecules to test as curvilinear objects sensibly extending according to a predetermined direction, said macromolecules presenting at least a sequence of target regions, each target region being associated with a tag and said macromolecules having underwent linearization according to said predetermined direction, wherein said method;
A module for generating a binary image from the sample image;
A module for calculating, for at least one template image, and for each sub-area of the binary image having the same size as the template image, a correlation score between the sub-area and the template image;
A module for selecting, for each sub-area of the binary image for which the correlation score with a template image is above a first given threshold, the corresponding sub-area of the sample image;
A module for calculating, for at least one reference code pattern, and for each selected sub-area of the sample image, an alignment score between the sub-area and the reference code pattern, said reference code pattern being defined by a given sequence of tags;
A module for identifying, for each selected sub-area of the sample image for which the alignment score with a reference code pattern is above a second given threshold, each target region depicted in said selected sub-area among the target regions associated with the tags defining said reference code pattern;
A module for outputting the different sequence(s) of identified target regions.

If configured for the second mechanism, the processor 11 implements:

A module for identifying a set of sequences of target regions on a plurality of macromolecules to test, from at least one sample image received from the scanner 2 connected to said equipment 10, said sample image depicting said macromolecules as curvilinear objects sensibly extending according to a predetermined direction, each target region being associated with a tag and said macromolecules having underwent linearization according to said predetermined direction;
A module for determining if there is at least one target region presenting a bimodal distribution of length as a function of a kurtosis value of the lengths of said target region;
A module for determining if there is at least one recurrent breakpoint position in said sequences of target regions;
A module for classifying the set of sequences of target regions as being abnormal if at least one target region presenting a bimodal distribution of length and/or at least one recurrent breakpoint position has been determined;
A module for outputting the result thereof.

Thus, the foregoing discussion discloses and describes merely exemplary embodiment of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, define, in part, the scope of the foregoing claim terminology.

1. A method of identifying at least one sequence of target regions on a plurality of macromolecules to test, each target region being associated with a tag and said macromolecules having underwent linearization according to a predetermined direction, wherein said method comprises performing by a processor (11) of equipment (10) the following steps:
a) receiving from a scanner (2) being sensitive to said tags, at least one sample image depicting said macromolecules as curvilinear objects sensibly extending according to said predetermined direction;
b) Generating a binary image from the sample image;
c) For at least one template image, and for each sub-area of the binary image having the same size as the template image, calculating a correlation score between the sub-area and the template image;
d) For each sub-area of the binary image for which the correlation score with a template image is above a first given threshold, selecting the corresponding sub-area of the sample image;
e) For at least one reference code pattern, and for each selected sub-area of the sample image, calculating an alignment score between the sub-area and the reference code pattern, said reference code pattern being defined by a given sequence of tags;
f) For each selected sub-area of the sample image for which the alignment score with a reference code pattern is above a second given threshold, identifying each target region depicted in said selected sub-area among the target regions associated with the tags defining said reference code pattern;
g) Outputting the different sequence(s) of identified target regions.

2. The method according to claim 1, wherein each target region is bound to a molecular marker, itself labelled with a tag.

3. The method according to claim 1, wherein the macromolecule is a nucleic acid.

4. The method according to claim 2, wherein the macromolecule is nucleic acid and wherein the molecular markers are oligonucleotides probes.

5. The method according to claim 4, wherein linearization of the macromolecule is performed by molecular combing or Fiber Fish.

6. The method according to claim 1, wherein said tags are fluorescent tags.

7. The method according to claim 1, wherein the target regions are associated with at least two different tags.

8. The method according to claim 7, wherein step (a) comprises, for a field of view of the scanner (2), receiving from the scanner (2) a sample image of the field of view for each tag.

9. The method according to claim 8, wherein step (b) comprises generating a binary image for each sample image, and merging the binary images from sample images of the same field of view.

10. The method according to claim 1, wherein said alignment score is computed using correlation method.

11. The method according to claim 1, wherein generating a binary image at step (b) comprises applying a local mean thresholding filter according to a direction which is orthogonal to said predetermined direction.

12. The method according to claim 1, further comprising a step (b′) of post-processing the generated binary image so as to remove unnecessary information.

13. The method according to claim 1, wherein the templates images of step (c) represent the same object according to different orientations.

14. The method according to claim 13, wherein said objet is a segment.

15. The method according to claim 13, wherein said different orientations are around said predetermined orientation.

16. The method according to claim 1, wherein step (d) comprises applying on the selected sub-areas a thresholding filter using machine learning algorithms.

17. The method according to claim 1, for detecting anomalies in the sequence(s) of identified target regions, further comprising:
Determining if there is at least one target region presenting a bimodal distribution of lengths of the said target region;
Determining if there is at least one recurrent breakpoint position in said sequences of target regions;
If at least one target region presenting a bimodal distribution of length and/or at least one recurrent breakpoint position has been determined, classifying the set of sequences of target regions as being abnormal, and outputting the result thereof.

18. A method of identifying at least one sequence of target regions on a plurality of macromolecules to test, each target region being associated with a tag and said macromolecules having underwent linearization according to a predetermined direction, wherein said method comprises performing by a processor (11) of equipment (10) the following steps:
a) receiving a plurality of candidate sub-areas of a sample image from a scanner (2) being sensitive to said tags, each sub-area possibly depicting one of said macromolecules as a curvilinear objects sensibly extending according to a predetermined direction;
b) applying on the candidate sub-areas a thresholding filter using machine learning algorithms so as to select relevant sub-areas;
c) For at least one reference code pattern, and for each selected sub-area, calculating an alignment score between the sub-area and the reference code pattern, said reference code pattern being defined by a given sequence of tags;
d) For each selected sub-area of the sample image for which the alignment score with a reference code pattern is above a second given threshold, identifying each target region depicted in said selected sub-area among the target regions associated with the tags defining said reference code pattern;
e) Outputting the different sequence(s) of identified target regions.

19. Equipment (10) comprising a processor (11) implementing:
a module for receiving from a scanner (2) connected to said equipment (10), at least one sample image depicting macromolecules to test as curvilinear objects sensibly extending according to a predetermined direction, said macromolecules presenting at least a sequence of target regions, each target region being associated with a tag and said macromolecules having underwent linearization according to said predetermined direction, wherein said method;
a module for generating a binary image from the sample image;
a module for calculating, for at least one template image, and for each sub-area of the binary image having the same size as the template image, a correlation score between the sub-area and the template image;
a module for selecting, for each sub-area of the binary image for which the correlation score with a template image is above a first given threshold, the corresponding sub-area of the sample image;
a module for calculating, for at least one reference code pattern, and for each selected sub-area of the sample image, an alignment score between the sub-area and the reference code pattern, said reference code pattern being defined by a given sequence of tags;
a module for identifying, for each selected sub-area of the sample image for which the alignment score with a reference code pattern is above a second given threshold, each target region depicted in said selected sub-area among the target regions associated with the tags defining said reference code pattern;
a module for outputting the different sequence(s) of identified target regions.
